(19) United States
(12) Patent Application Publication
(10) Pub. No.: US 2008/0095306 A1
(43) Pub. Date: Apr. 24, 2008

(54) SYSTEM AND METHOD FOR PARAMETER SELECTION FOR IMAGE DATA DISPLAYS

(76) Inventors: Vernon Thomas Jensen, Draper, UT (US); Joel Frederick Zuhars, Haverhill, MA (US)

Correspondence Address: RAMIREZ & SMITH, PO BOX 341179, AUSTIN, TX, 78734, US

(21) Appl. No.: 11/550,727

(22) Filed: Oct. 18, 2006

(51) Int. Cl. A61B 6/00

(52) U.S. Cl. 378/10

(57) ABSTRACT

A user interface is provided for manipulation of image data. In some embodiments, the interface includes a display region spanning only a locus of interdependent variable values that is exclusive of invalid pairs of parameter values.

### FIELD OF THE DISCLOSURE

This disclosure relates generally to visualizing and selecting parameters for image data displays, and in particular to a system and method for visualizing and selecting window and level parameters for image data displays.

### BACKGROUND

Many medical diagnostic, surgical and interventional procedures rely on non-invasive or minimally invasive tools to provide information, often in the form of images, descriptive of status of internal portions of anatomy or organs of a patient. Some of these tools include thermal imaging (e.g., mammography), ultrasonic probes, magnetic resonance (MR), positron emission tomography (PET), computed tomography (CT), single photon emission-computed tomography (SPECT) and optical imaging and/or X-ray radiation based techniques. In some instances, imaging aids, such as contrast-enhancing agents, are introduced into the subject or patient to aid in increasing available data content from the imaging technique or techniques being employed.

Each of these tools presents advantages in particularized situations, has technological limitations, may require set-up and analysis time, can include risks and also has associated costs. As a result, a cost-benefit analysis that also reflects the degree of urgency with respect to a particular diagnostic trajectory often favors usage of X-ray radiation-based measurement techniques.

Several factors influence image quality resulting from an X-ray radiation procedure. Statistical photon noise resulting from characteristics of the X-ray radiation source and the X-ray radiation generation conditions tend to dominate other noise sources in formation of an X-ray radiation-based image. Signal conditioning consistent with achieving suitable contrast between various image portions, and contrast enhancement techniques, are also important considerations in providing diagnostic images, and these issues require increasingly sophisticated treatment as dose and/or photon energy are decreased.

One of the key tenets of medical X-ray radiation imaging is that image quality should be carefully considered in determining exposure conditions. Exposure considerations include predetermined dose criteria relating to the amount of X-ray radiation delivered to a test subject or patient in order to provide images. The design and operation of a detector used for medical X-ray radiation imaging should therefore be tailored, responsive to the particularized task and measurement conditions, including variables in test subject mass, radio-opacity and the like, to provide high image quality for each X-ray radiation exposure that is incident on the detector.

Many new imaging tools employ pixelated X-ray radiation detectors (detectors comprising a geometric array of multiple detector elements, where each detector element may be individually representative of at least a portion of a picture element or pixel in the resultant image). Among other things, pixelated detectors facilitate digital representation of images and other data resulting from usage of the systems, which, in turn, enables digital signal processing, and digital data storage and data transmission technologies.

As these new imaging tools and enhancements have been developed and combined, providing synergistic benefits, the volume of data resulting from an imaging procedure has grown, in tandem with the increasing gamut of capabilities for analyzing, displaying and employing the data. As a result, it is increasingly difficult and time-consuming to examine the many elements of information resulting from an imaging procedure in order to determine and select the vital few elements needed for various highly specialized types of procedures. In turn, this explosion of data results in delay in applying the results from the procedure, and this is particularly felt in situations requiring extremely rapid response, for example, during surgery, or responsive to unexpected demand for medical services, such as an influx of multiple critically-injured patients following one or more traumatic events such as vehicular disasters and the like.

Digital images are made up of pixels, and these images are generally visualized by assigning each pixel a numerical value corresponding to a color, typically a shade of gray, and then displaying that color in the corresponding position for that pixel on a graphical display. A digital image can be adjusted by varying the numerical values of each pixel. The raw image data is manipulated by software using algorithms and mathematical computations to optimize the image. However, once the image is displayed, it can be further processed by the operator to change parameters as desired.

One method by which the pixels of an image can be assigned color values for display purposes is to map each pixel intensity value, or brightness, to a particular shade of gray, based on window and level parameter settings. The window parameter setting determines how large the radiodensity range of pixel intensities will be, in the mapping from white to black, with intensities outside the range being uniformly set to either white or black. A large window setting will cause a large range to be displayed simultaneously, but with less differentiation between values within the range. A small window setting will cause a small intensity range to be displayed in the image with higher differentiation. The level parameter setting sets the intensity level which is the midpoint of the displayed range. Raising and lowering the level setting causes different effective ranges to receive the detail.

Processing or filtering image data with window and level parameters is well known. The window and level parameter values deterministically specify the filter characteristics. Also, particular pairs of window and level parameter values are known to generate a filtered image that represents particular types of anatomy in medical images of certain modalities when the modality image voxel to anatomy density is standardized.

Using prior art methods, valid ranges of the window and level parameters are not independent of the values of the parameters themselves, unlike brightness and contrast filtering. If the window parameter is at a theoretical maximum value, there is only a single legitimate level value, that being in the middle of its potential range, and if the level parameter is not in the middle of its potential range, the window parameter is limited to the width of the window that would cause one edge of the window to be at either its theoretical minimum or maximum value. Therefore, the use of independent controls, such as sliders for each parameter that go from a fixed minimum value to a fixed maximum value, for selecting the window and level parameters can lead to invalid or non-deterministic results.

For the reasons stated above, and for other reasons discussed below, which will become apparent to those skilled in the art upon reading and understanding the present disclosure, there are needs in the art to provide more highly automated image computation engines and protocols for application and usage of such capabilities, in order to streamline gathering of information in support of increasingly stringent and exacting performance and economic standards in settings such as medical imaging.

### BRIEF DESCRIPTION

The above-mentioned shortcomings, disadvantages and problems are addressed herein, which will be understood by reading and studying the following disclosure.

In one aspect, a system having a user interface is provided for manipulation of image data. The system includes a user interface having a display region spanning only a locus of interdependent variable values that is exclusive of invalid pairs of parameter values.

In another aspect, a method for adjusting a filtering function is described. The method includes displaying a user interface for manipulation of image data in an imaging system, and providing a display region within the interface, the display region spanning only a locus of interdependent variable values exclusive of invalid groups of parameter values.

In a further aspect, an article of manufacture forming a computer-readable medium having computer-readable instructions embodied thereon is disclosed. The instructions, when executed by one or more processors, cause the one or more processors to perform acts of generating a user interface for manipulation of image data and providing a display region within the user interface. The display region spans only a locus of interdependent variable values that is exclusive of invalid groups of parameter values.

Systems, processes, and computer-readable media of varying scope are described herein. In addition to the aspects and advantages described in this summary, further aspects and advantages will become apparent by reference to the drawings and by reading the following detailed description.

### BRIEF DESCRIPTION OF THE DRAWINGS

FIG. 1 is a block diagram of an overview of a system configured to improve the display of images from an imaging apparatus.

FIGS. 2 through 7 illustrate simplified examples of user interfaces capable of utility in the system of FIG. 1.

FIG. 8 is a flowchart describing a process capable of utility in the system of FIG. 1.

FIG. 9 illustrates an example of a general computation resource useful in the context of the environment of FIG. 1.

### DETAILED DESCRIPTION

In the following detailed description, reference is made to the accompanying drawings that form a part hereof, and in which are shown, by way of illustration, specific embodiments that may be practiced. These embodiments are described in sufficient detail to enable those skilled in the art to practice the embodiments, and it is to be understood that other embodiments may be utilized, and that logical, mechanical, electrical and other changes may be made, without departing from the scope of the embodiments.

As used herein, the term “illumination” refers to exposure to photons, electromagnetic radiation, X-ray radiation, phonons (e.g., insonification via ultrasound) or other wave phenomena, which do not necessarily correspond to light visible to humans. Ranges of parameter values described herein are understood to include all subranges falling therewithin. The following detailed description is, therefore, not to be taken in a limiting sense.

The detailed description is divided into five sections. In the first section, a system level overview is provided. In the second section, examples of user interfaces are described. In the third section, a process capable of utility with the system is discussed. The fourth section discloses hardware and an operating environment, in conjunction with which embodiments may be practiced. The fifth section provides a conclusion which reviews aspects of the subject matter described in the preceding four segments of the detailed description. A technical effect of the systems and processes described herein includes multiple and complementary capability for visualization and selection of parameters for displaying images, responsive to user input instructions.

### I. System Overview

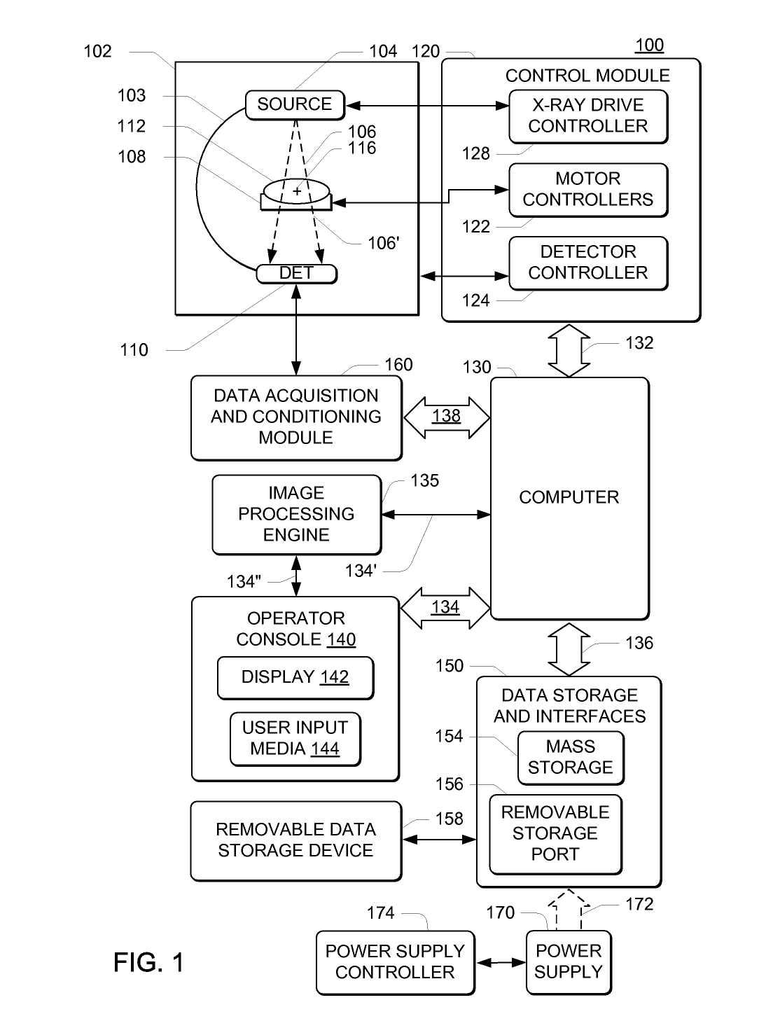

FIG. 1 is a simplified diagram of an overview of a modified system 100 configured to improve the display of images from an imaging apparatus. The system 100 optionally includes an imaging apparatus 102 including a gantry, C-arm or other support 103 for an illumination source 104, such as an X-ray radiation illumination source, capable of providing illumination 106, such as X-rays or other imaging illumination, and may optionally include a test subject support 108 that is transmissive with respect to the illumination 106 and that is positioned above a detector 110 that is also opposed to the illumination source 104.

In one embodiment, components of the system 100 and a test subject 112 are maintained in a defined geometric relationship to one another by the gantry/c-arm 103. A distance between the illumination source 104 and the detector 110 may be varied, depending on the type of examination sought, and the angle of the illumination 106 respective to the test subject 112 can be adjusted with respect to the body to be imaged responsive to the nature of imaging desired.

In one embodiment, the test subject support 108 is configured to support and/or cause controlled motion of the test subject 112, such as a living human or animal patient, or other test subject 112 suitable for imaging, above the detector 110 so that illumination 106′ is incident thereon after passing through the test subject 112. In turn, information from the detector 110 reveals internal aspects of the test subject 112. In some modes of operation, such as CT, the gantry 102 or C-arm 103 and test subject support or table 108 cooperatively engage to move the test subject 112 longitudinally, that is, along an axis extending into and out of the plane of FIG. 1. In some modes of operation, the gantry 102 rotates the X-ray radiation source 104 and detector 110 about an axis 116, while the support 108 moves longitudinally to provide a helical series of scans of the test subject 112, where a pitch of the helices is defined as a ratio of a longitudinal distance traveled by the table 108 during a complete revolution of the gantry 102, compared to a length of the detector 110 along the axis 116 of linear motion.

In one embodiment, the detector 110 comprises a floating receptor, that is, a detector 110 that is not coupled to a gantry or C-arm 103 and that is not associated with a patient table 108. In other words, the floating receptor digital detector 110 is portable and is hence ‘floating’ with respect to other elements of the system 100, and it is attached to the rest of the system via a tether or a wireless communication system. The term ‘floating’ is meant to indicate that its position is completely subject to the user and is not controlled via a gantry, table or other system device.

In one embodiment, the floating receptor 110 may be positioned opposite the source 104 with the test subject 112 being located between the source 104 and the floating receptor 110, by placing the floating receptor beneath the test subject 112, for example. In one embodiment, the detector 110 may be an image intensifier based imaging system. In one embodiment, the detector 110 may be large enough to capture a full-sized image of the test subject 112, or may comprise Apollo-like or “full body” digital detector panels, which may eliminate need for longitudinal motion of the support 108.

The system 100 also optionally includes a control module 120. The control module 120 may include a motor controller 122 configured to move the test subject support 108 and thus the test subject 112 relative to the illumination source 104 and/or detector 110, and may also control motors in the gantry 102, C-arm 103 or other device and/or operate to position/move the illumination source 104 relative to the test subject 112 and/or the detector 110.

The control module 120 may include a detector controller 124 configured to control elements within the detector 110 and to facilitate data transfer therefrom. The control module 120 may also include a drive controller 128 configured to control electrical drive parameters delivered to the illumination source 104. One or more computers 130 are connected to the control module 120 via a bus 132 configured for receiving data descriptive of operating conditions and configurations and for supplying appropriate control signals. Buses 134 and 134′ act to transfer data and control signals, for example with respect to an image processing module 135, via interconnections such as 134′, 134″ that are configured for exchange of signals and data to and/or from the computer 130 as well as other elements of the system 100 and/or external computation or communications resources.

The system 100 also includes a bus 136, a bus 138 and an operator console 140. The operator console 140 is coupled to the system 100 through the bus 134. The operator console 140 includes one or more displays 142 and a user input interface 144. The user input interface 144 may include a keyboard, touchscreen, mouse or other tactile input device, capability for voice commands and/or other input devices. The one or more displays 142 provide video, symbolic and/or audio information relative to operation of system 100, displaying user-selectable options and images descriptive of the test subject 112, and may display a user interface (e.g., see Section II, infra) for facilitating user selection among various modes of operation and other system settings.

The image processing module 135 facilitates automation of accurate measurement and assessment, and is capable of forming multiple, coordinated images for display, for example via the displays 142. The image processing module 135 may comprise a separate and distinct module, which may include application-specific integrated circuitry, or may comprise one or more processors coupled with suitable computer-readable program modules, or may comprise a portion of the computer 130 or other computation device.

The system 100 also includes data storage and memory devices 150, coupled via the bus 136 to the computer 130 through suitable interfaces. The data storage and memory devices 150 include mass data storage capabilities 154 and one or more removable data storage device ports 156. The one or more removable data storage device ports 156 are adapted to detachably couple to portable data memories 158, which may include optical, magnetic and/or semiconductor memories and may have read and/or write capabilities, and which may be volatile or non-volatile devices or may include a combination of the preceding capabilities.

The system 100 further includes a data acquisition and conditioning module 160 that has data inputs coupled to the detector 110 and that is coupled by the bus 138 to the one or more computers 130. The data acquisition and conditioning module 160 includes analog to digital conversion circuitry for capturing analog data from the detector 110 and then converting those data from the detector 110 into digital form, to be supplied to the one or more computers 130 for ultimate display via at least one of the displays 142 and for potential storage in the mass storage device 154 and/or data exchange with remote facilities (not shown in FIG. 1). The acquired image data may be conditioned in the data acquisition and conditioning module 160, the image processing module 135, the one or more computers 130, or a combination thereof.

The system 100 also includes a power supply 170, coupled via interconnections represented as a power supply bus 172, shown in dashed outline, to other system elements, and a power supply controller 174. The full range of interconnection of the power supply 170 to other elements of the system 100 is not shown in FIG. 1, in order to promote simplicity of illustration and ease of understanding.

In some embodiments, the system 100 is configured to be a mobile system equipped with a portable power supply 170, such as a battery. In other words, the system 100 may comprise a wheeled unit and may be electromotively powered in self-contained fashion, lending physical agility to the ensemble of attributes offered by the system 100.

In some settings, such as in an emergency room, articulation of a mobility function may be limited to motion of a system 100 that is generally dedicated to application within that setting, suite or environment. In other settings, such mobility may include scheduled sequential visits to areas such as a cardiac unit, an ICU and other loci, where such imaging capability provides critical assistance, such as when the test subject 112 is not postured in a fashion consistent with movement of the test subject 112 and yet aperiodic variations in work load are not favorable to cost-effective deployment of a system 100 incapable of ready, self-propelled, operator-guided, “at need” physical translation of location. In one embodiment, electrically-powered motors coupled to a drive train effectuate operator-directed motion of the system 100.

Self-portable systems 100 employing a C-arm 103, rather than a gantry 102, also provide motion capabilities relative to the test subject 112 and promote known spatial relationships between the illumination source 104 and the detector 110. Other types of multidimensional data collection techniques, employing fan beams, cone beams and the like also may be employed for imaging, together with detectors ranging in size up to a size sufficient to collect x-ray radiation over the entire test subject 112.

In some deployment scenarios, one or more portable systems 100, may be kept in a “corral” adjacent a series of operating suites, and be called upon in the course of surgery in order to provide the surgeon with live, “on the spot” information regarding the procedure and the patient/test subject 112. In these situations, time is often of the essence, for multiple reasons: it is desirable to keep exposure of the patient 112 to X-rays low; multiple surgical suites often rely on relatively few systems 100; and the fact that it is generally desirable to conclude surgery rapidly, to reduce bleeding, as well as to reduce need for anesthesia (for example, the length of time a patient 112 is under general anesthesia). As a result, there are benefits to equipping the system 100 with intuitive, easy-to-operate, interface tools that readily promote rapid, intuitive selection of settings appropriate to a specific situation, and which also provide increased contrast between portions of the region of interest over a suitable span of radiographic densities.

As part of initiating data collection, and then in the subsequent process of analyzing the data from the system 100, a clinician will need to interact with the system 100 in order to select a measurement type and to specify data manipulation and display aspects. Conversion of data from the detector 110 to diagnostically-useful image data includes specification of settings appropriate to the desired type of image and to aspects specific to the individual patient 112, which may include selecting a filter function and parameters for fitting that selected filter function to various characteristics present in the data.

It is possible that the graphical technique and parameter selection may be executed on the same physical system that controls the source 104 and other elements of the system 100 that effectuate the x-ray exposure etc., and this may be desirable, for example, in the operating room. However, another manner in which this technique may be employed include transfers, through physical or electronic media, of the Hounsfield unit voxel data from the x-ray exposure aspect of the imaging procedure to a remote computing device where the technique may be applied on the transferred data, independently of whether or not the disclosed technique or a different technique was applied on the scanner system itself as part of the scan data collection. This latter situation may apply with respect to diagnostic x-ray procedures, for example, where time is not of the essence.

Brightness and contrast and window and level filtering exemplify alternative data filter techniques useful in image formation, as well as with other multidimensional/volumetric imaging approaches, such as three-dimensional cone-beam back reconstruction/projection, segment reconstruction methodologies, back-projection schemes, and others, in conjunction with appropriately selected mathematical data treatment tools. An extremely rich panoply of such algorithms and methodologies have been developed in broadly varying contexts, ranging from radio astronomy to seismic/geophysical investigations, spread-spectrum communications techniques and many other data intensive arenas. Adaptation of such data extraction/enhancement tools from an initial context of application to other contexts is a relatively mature area of endeavor.

In medical imaging using datasets acquired via controlled and known motion of an illumination source 104 and a multi-element detector 110 relative to an object of study 112 to provide a series of “snapshots” including information descriptive of volumetric information, filtering is a tool that finds utility. For example, brightness and contrast filtering involves user adjustment of two independent parameters, while window and level filtering is implemented via selection of a pair of parameter values, where the parameters are interdependent. As a result, parameter values for brightness and contrast filtering that are specific to the particular type of anatomy and image may be independently specified by the clinician, without necessarily risking selection of non-deterministic value pairs. However, in window and level filtering, interdependency of the user-adjustable parameters can lead to invalid results, when the parameter values are independently specified.

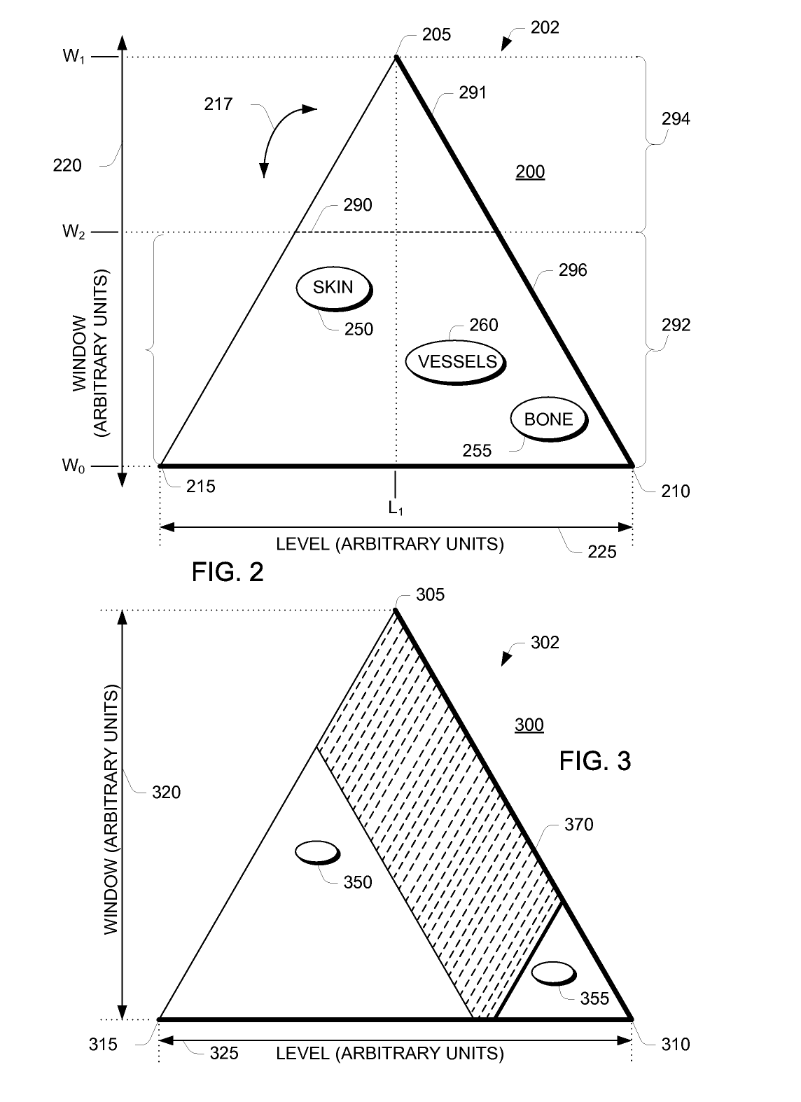

Consequently, conventional filter parameter selection tools, such as separate scroll bars or sliders corresponding to the parameters, may present problems when used with window and level filtering. The graphical user interfaces of FIGS. 2 through 7, described below in more detail with reference to Section II, provide intuitive techniques and tools for selection of valid parameter pairs when the parameters are not independent.

### II. Exemplary User Interfaces

FIGS. 2 through 7 are simplified illustrations of exemplary user interfaces 200 through 700, respectively, capable of utility in the system 100 of FIG. 1 for parameter selection and in specifying a filter function for a particular data set or imaging task. The user interfaces 200 through 700 find particular application in situations involving selection of a valid pair of parameters, when multiple mutually-dependent parameters specify values related to appropriate image formation and display.

In FIG. 2, the user interface 200 includes a trigonal display field 202 having apices 205 (top vertex, corresponding to a value denoted W₁), 210 (lower right-hand vertex) and 215 (lower left-hand vertex). The representation of the user interface 200 of FIG. 2 is presented as an equilateral triangle or equiangular figure, however, it will be appreciated that numerous other exemplars encompass the teachings of the present disclosure and provide utility. Other forms of polygonal or curvilinear representations, or other angular dispositions, do not depart from the scope of the disclosed subject matter.

It will be appreciated that any angular orientation of the user interface 200 accomplishes the same purposes, as indicated by bidirectional arrowed arc 217, and the same is true for any other configuration or adaptation of the teachings of the present disclosure. The user interface 200 is adapted in conformance with axes representing interdependent variables. An ordinate 220, (e.g., a first interdependent variable) has values W₀, W₁ and W₂ noted thereon, is labeled WINDOW (ARBITRARY UNITS) and corresponds to a WINDOW variable. An abscissa 225 (e.g., a second interdependent variable) is labeled LEVEL (ARBITRARY UNITS) and corresponds to a LEVEL variable, in this example. The ordinate 220 and abscissa 225 are illustrated for explanatory purposes.

With respect to all of the user interfaces 200 through 700 of FIGS. 2 through 7, respectively, when the WINDOW parameter attains a theoretical maximum value (e.g., W₁), there is only one valid LEVEL value, i.e., at a midpoint of the potential range (value L₁). As the WINDOW variable decreases through the range of possible or applicable values towards W₀, the corresponding range of deterministic LEVEL variables increases linearly.

In this range of LEVEL values, when the LEVEL parameter is not in the middle of its potential range (i.e., is not in the area of the value denoted L₁), the WINDOW parameter is limited to values within the width of the window that would cause one edge of the window to be at either the theoretical minimum or maximum LEVEL value. Therefore, the use of independent controls, such as scroll bars or sliders, to independently select a value for each parameter, with each control spanning a range extending from a fixed minimum to a fixed maximum value for the WINDOW and LEVEL parameters, can lead to invalid or non-deterministic results.

The user interfaces 200 through 700 of FIGS. 2 through 7 exemplify features intended to facilitate graceful and intuitive user interaction in fulfilling several different functions, in an integrated manner. As an example, the display field 202 of FIG. 2 shows locus 250 (labeled SKIN), locus 255 (labeled BONE) and locus 260 (labeled VESSELS), each disposed within a particularized area interior to the trigonal display field 202.

FIG. 2 also shows a horizontal line 290 corresponding to WINDOW parameter value W₂, and thus separating a region 291 from other portions of the display field 202. In some situations, a finer degree of display resolution or granularity may be desired. It may also be known that the parameters of interest fall into a definable portion, such as that corresponding to a range 292 bounded by values W₂ and W₀. For example, it may be known that a portion 294 of the display range, extending from the value W₁ to the value W₂, is blocked, as described below with reference to FIGS. 3 through 7.

In such a situation, where only a lower region 296 within the range 292 of the display field 202, extending from the value W₂ to the value W₀, is of interest, it may be desirable to modify the shape of the display field 202. Elimination of the portion 291 spanning the range 294 and forms frustum 296 having top edge 290. The frustum 296 then may be enlarged to occupy all of the monitor 142 or display real estate allocated to the display field 202. It will be appreciated that the truncation need not correspond to a horizontal line.

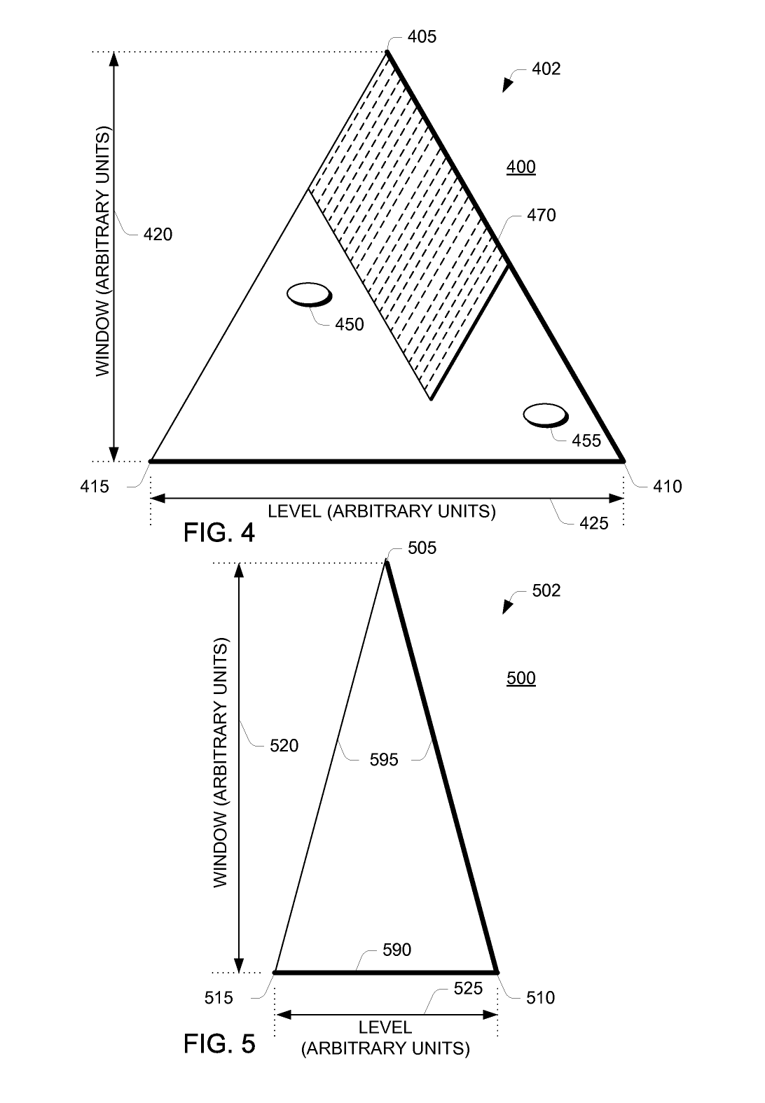

There are also advantages that can be derived from blocking certain density or level ranges from being within the window filter. For example, due to various image-processing artifacts, the presence of objects of distinctly different opacity often degrades the desired anatomical image quality significantly. An approach to addressing such artifacts is described below with reference to FIGS. 3 and 4.

When, for example, it is known that metal of a certain radiodensity range is present in the arena being imaged, blocking out that range in an intuitive manner allows the clinician to easily explore the WINDOW/LEVEL parametric surface, near the blocked range, without including data from the blocked range in forming the desired image. This may usefully be represented as an excluded region, and this aspect is described below with reference to FIGS. 3 and 4. The clinician could also select multiple available sections within the segmented region or triangle to create a windowing function (see FIG. 6) spanning the blocked range without including the blocked range.

In many situations involving need to distinguish between tissues having relevant features corresponding to a relatively narrow range of radio-opacities, such as visualization of vasculature, it is often the case that only a limited range of the possible range of gray scale values represent the relevant anatomical aspects within the field of interest. In other words, when the values representing pixel luminance data from information which the detector 110 is able to provide range from zero, representing complete radio-opacity or little or no transmissivity to X-rays (e.g., bone or metallic objects), to one hundred, representing complete radio-transparency to X-rays (e.g., air), it may be the case that the anatomical features of interest provide values only over a range of, for example, twenty to seventy.

In that situation, it can be advantageous to remap the range of gray scale values representing the desired image data over a range of zero to one hundred, in order to increase useful levels of contrast in the resulting image, promoting visual distinction between the images of the different anatomical elements within the region of interest. However, when another object or feature, for example a metallic object, also falls within the region of interest, and provides an anomalous gray scale value, perhaps one corresponding to a radio-opacity associated with a gray scale value of five, the remapping would map the range of five to seventy over the display pixel luminance range of zero to one hundred. This would result in losing a great deal of contrast, due to inclusion of outlying data values corresponding to the metallic object, thus tending to obscure the desired contrast between the portions of interest.

By blocking certain values, such as, in this example, zero to twenty, the resulting image data is processed to be displayed such that a pixel having a value of twenty would be black (or set to zero on the display gray scale) and a pixel having a value of seventy would be white (or set to a value of one hundred). In this scenario, the radio-opaque object (e.g., the metallic object resulting in the gray scale value of five) would still show on the image as a black region. A technique for effectuation of this type of blocking is described below with reference to FIGS. 3 and 4.

In FIG. 3, a user interface 300 includes a display field 302 having apices 305, 310 and 315. The display field 302 is illustrated in conjunction with ordinate 320 and abscissa 325, for explanatory purposes.

FIG. 3 also depicts a locus 350, analogous to the locus 250 of FIG. 2, and a locus 355, corresponding to the locus 255. However, the exemplary display field 302 does not include text labels, and the loci 350 and 355 present smaller “footprints” than their counterparts in FIG. 2. Such areal options may be included as clinician preferences, or as application-specific selection aspects, and are generally independent of identificatory aspects. Put another way, larger or smaller predefined zones or regions or loci of interest may be employed.

In the example of FIG. 3, an area 370 representing a blocked region is illustrated as being bounded via linear segments, for simplicity of illustration and ease of understanding, however, it will be appreciated that other (e.g., curvilinear segments etc.) border shapes may find utility. The bounded excluded area 370 blocks a portion of the display field 302, including that corresponding to the locus 260 of FIG. 2.

In FIG. 4, a user interface 400 includes a display field 402 having corners 405, 410 and 415. The display field 402 is depicted together with ordinate 420 and abscissa 425, for explanatory purposes. The display field 402 includes area 450 (analogous to locus SKIN 250) and area 455 (analogous to locus BONE 255). Area 470 represents a blocked region, as described above, but corresponding to a smaller portion of the display field 402 than the blocked region 370 of FIG. 3.

In FIG. 5, a user interface 500 includes a display field 502, shown as a triangle having cusps 505, 510 and 515. Ordinate 520 and abscissa 525 are included in FIG. 5 for illustrative purposes. The display field 502 forms an isosceles triangle.

In other words, the display field 502 has base 590 that is shorter than a length of sides 595, conserving display real estate, without necessarily incurring resolution capacity compromise. As a result, display of multiple images, or of multiple selected image portions, is facilitated. Additionally, the display field 502 allows more of the overall display real estate to be devoted to other relevant data, such as magnified regions of interest, when the display field 502 is combined with zoom-in or expansion capacities.

A traditional window and level display could also be provided, as in FIG. 6, which shows the window of displayed pixel densities in the form of a filter. This display would update to reflect the selection(s) made in the triangle display, and would provide a better understanding of the mapping between the triangle display and the filter display for users that are more familiar with the latter.

In FIG. 6, a user interface 600 includes a display field 602 having apices 605, 610 and 615. The display field 602 is shown adjacent horizontal axis 620 and vertical axis 625, for explanatory purposes. A first region of interest 650, and an expanded boundary 652 for the first region 650, are shown at the left side of the display field 602. A second region of interest 660, and an expanded corresponding inset 662, are depicted at the right side of the display field 602. These are but two different ways, of many possible ways, in which “zoom” features may be provided. A blocked region 670 is illustrated between the regions 650 and 660.

The disclosed visual displays or user interfaces may also include one or more magnification scaling features. For example, a clinician may interact with the system 100 via a touchscreen configured to render an image within the display 142, where the image includes a plurality of predetermined loci each having a defined radius (e.g., such as SKIN 250, BONE 255, VESSELS 260, FIG. 2, or loci 350 and 355 of FIG. 3).

In other words, each individual pixel of the display represents a unique window/level parameter pairing. Therefore, the defined radius or locus is a representation of a set of individual window/level parameter pairings that are likely to generate an image that adequately represents the indicated anatomical type.

Thus, the defined radius or locus corresponds to a group of such parameter pairs, amongst which the user can select to provide a relatively idealized image representation, with the selection being possible via various means, such as by sliding a finger across the region (e.g., 660) or an expanded counterpart region (e.g., 662) corresponding to the defined radius or locus, which may be displayed on a touchscreen monitor 142.

The clinician may opt to switch between the representations 660 and 662 in order to use a single input mode for selection, or multiple or alternative tools may be used to designate a particular element. For example, the clinician may select a region of interest such as the locus 660 using a finger, and then tap the touchscreen 142, or use a voice command, or otherwise activate a switch function, and then use the same finger to effectuate the fine tuning within the expanded locus 662. As another example, the clinician may use a finger to select a locus such as 660, while the clinician employs a mouse or other tool, for example by using the other hand, to select a particularized point within the resulting expanded zone 662, etc. These approaches allow portions of display real estate not required for the display field 602 to be employed in order to render an enlarged inset image, e.g., an oblate elliptic or otherwise-shaped locus 662.

It may be desirable to facilitate fine tuning of the window/level parameter pair selection corresponding to one or more regions of high anatomical interest. This may also be effected by warping an interior portion of the display field 602 about the region of interest, essentially creating a non-linear level scale.

In one embodiment, the non-linear or expanded display portion is dynamically updated, based on the most recent selection. For example, when a touchscreen is employed as the display 142 of FIG. 1, the size of the clinician's fingertip may be inconsistent with the level of granularity needed in order to accurately select (“fine-tune”) the desired parameters. In this example, warping of a selected portion of the display field 602, resulting in an expanded (“fish-eye”) view, represented, for example, by the region 652, may follow the clinician's finger on the display 142, creating an effect such as the clinician's finger moving a fish-eye lens around within the display field 602.

Also, within the user interfaces 200 through 600, parameter value pairs typically representing specific anatomy types, such as skin, bone, etc., may be indicated by a point and label, an icon, or any other method for indicating a particular standardized position or zone within the display to the clinician. However, these ‘standard’ indications may not be completely appropriate for a particular image. The disclosed user interfaces 200 through 600 allow the clinician to easily ‘search’ for a better value pair to filter the particular image data by indicating positions near the ‘standard’ position, either by clicking around it with a mouse or other tactile pointer, or by dragging a finger around the region of interest on the display while watching continuously-updated images, or via any other appropriate method.

In one embodiment, a nonlinear or “zoom in” display capability may be invoked when an element (such as a finger) encroaches on or engages one of the predetermined loci, with the result that a localized portion of the image extending about the element is displayed on a scale enlarged in comparison to a remainder of the image. This capability allows clinicians having different sizes of fingers to equally easily access even relatively small portions of the range encompassed by user interfaces 200 through 600.

The example of FIG. 6 also includes a graphical display 680 of an density and amplitude transfer function for mapping from density to image pixel grayscale levels, in a form corresponding to a well-known, but somewhat awkward, technique, for selection of window W and level L values. That approach also allows independent adjustment of interdependent variables, which can result in non-deterministic value pairs being selected, as was described in part with reference to FIG. 2.

The graphical display 680 facilitates usage of the user interface 600 for clinicians whose experience with prior art systems lends bias towards those models. A locus 676 within the display field 602 corresponds to a level value L, denoted via the reference character 686 shown with respect to the graphical display 680, and a window value W.

The level value L corresponds to a midpoint of the window function, which, in the example given above with reference to FIG. 1, with grayscale units ranging from twenty to seventy, corresponds to a level value L of forty-five. For the same example, the window width value W corresponds to a span of fifty.

The graphical display 680 also includes ordinate 682, labeled GRAYSCALE (ARBITRARY UNITS) and abscissa 685, labeled DENSITY (ARBITRARY UNITS). A piece-wise linear representation 687 having a span 689 denoting width W of the window function is provided with the graphical display 680.

Graphical displays 680 of the type shown with respect to FIG. 6 may, at times, suffer one or more deficiencies when employed in conjunction with selection of pairs of mutually-dependent variables. One such issue reflects the fact that such displays 680 are typically manipulated via independent horizontal and vertical axis control functions relating to the interdependent parameters, and thus presents opportunity for the clinician to select parameter pair values inconsistent with accurate or effective usage of the resulting image.

The disclosed user interfaces represent the full range of appropriate or legitimate parametric value pairs for WINDOW/LEVEL selection, and do not allow any invalid parametric value pairs to be selected. In one embodiment, extrema 205, 210, 215, . . . , 615 of the user interfaces 200 through 600 represent the following specific pairs of window and level values:

1) At vertices 205, 305, 405, 505, 605, the WINDOW parameter has its maximum value (W₁ in FIG. 2), and the LEVEL parameter is its mid-range value (L₁ in FIG. 2).

2) At vertices 210, 310, 410, 510, 610, the WINDOW parameter is zero (W₀ in FIG. 2), and the LEVEL parameter has its maximum value.

3) At vertices 215, 315, 415, 515, 615, the WINDOW parameter is zero (W₀), and the LEVEL parameter has its minimum value.

Value pairs for all other points on, or within, the display fields shown in FIGS. 2 through 6 can then be extrapolated from these values. The height of the triangles 200 through 600 represent the WINDOW value from a minimum, such as 0 (adjacent the axes 225 through 665, in these examples), to a maximum (at the apices 205 through 605, or to the dashed line 205′).

The side-to-side position within the triangles 202 through 602 represents the LEVEL value over a range extending from its minimum (left-hand edge) to its maximum (right-hand edge). A value pair is selectable by the clinician via a mouse point and click, a physical touch on a touchscreen monitor, a joystick, or any other process for indicating a position on graphic displays, such as the display 142 of FIG. 1.

The teachings of the present disclosure may also be applied as part of a more general user interface, in which the triangular (or otherwise-shaped) border would not be explicitly displayed, but instead, the responsive region of a typically rectangular display window could be limited to a suitable shape, without necessarily explicitly displaying boundaries of that shape.

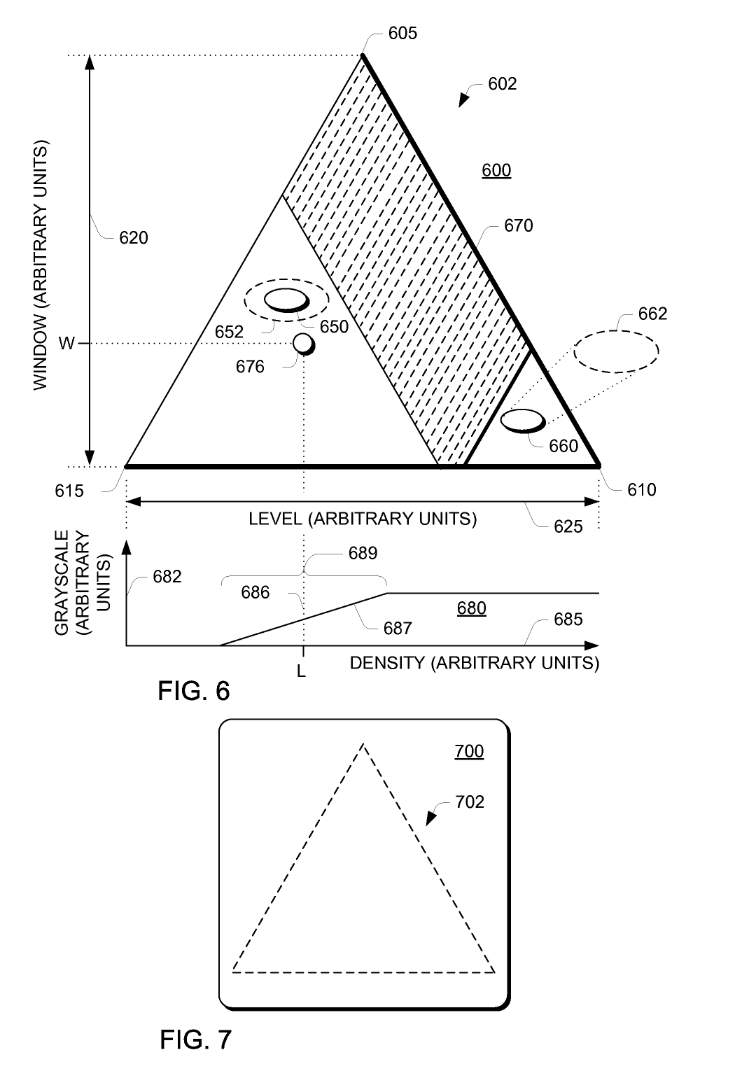

FIG. 7 illustrates a user interface 700 that includes a triangular active zone 702. The embodiment shown in FIG. 7 illustrates a way in which the disclosed concepts may implemented as part of a window interactor, where the outline of the active region is not explicitly displayed. Instead, the active or responsive region 702 within the user interface 700 may be limited, for example, to a triangular shape, as shown in dashed outline in FIG. 7.

While all potential orientations of the displayed triangles of the user interfaces 200 through 700 are included within the scope of the present disclosure, the upwards-pointing triangular orientations shown in FIGS. 2 through 7 provide useful and intuitive examples.

Also, within the displayed triangles 200 through 600 the value pairs that typically represent anatomy types, such as, for example, the loci 250 and 350 corresponding to SKIN, or the loci 255 and 355 relating to BONE, of FIGS. 2 and 3, respectively, etc., may be indicated with one or more of: a point; a text label, an area, a pictographic display, an icon, or any other mode for indicating a particular position within the displayed shape to the clinician. An advantage for the clinician, in these examples, is that prior art or ‘standard’ indications may not be completely accurate for a particular image, and the disclosed user interfaces 200 through 600 allow the clinician to easily ‘search’ for an improved value pair relative to the predetermined representative value pair for that tissue type, to filter the particular image by indicating/selecting positions around the ‘standard’ position, for example, by clicking around it with a mouse pointer, or by dragging a finger around it on the display 142, while watching the continuously updating filter results, or any other appropriate usage approach.

As a result, the examples of Section II describe various control and image processing options which are available, each presenting strengths in particularized situation. These may be structured to facilitate user input via a tactical input-output device to adjust views via user interfaces 200 through 600 of FIGS. 2 through 6, respectively, as described above. Examples of tactile input/output media include touchscreens, keyboards, switchable rollerball devices, and the like. As well, voice recognition and other forms of input-output functionality may be enabled.

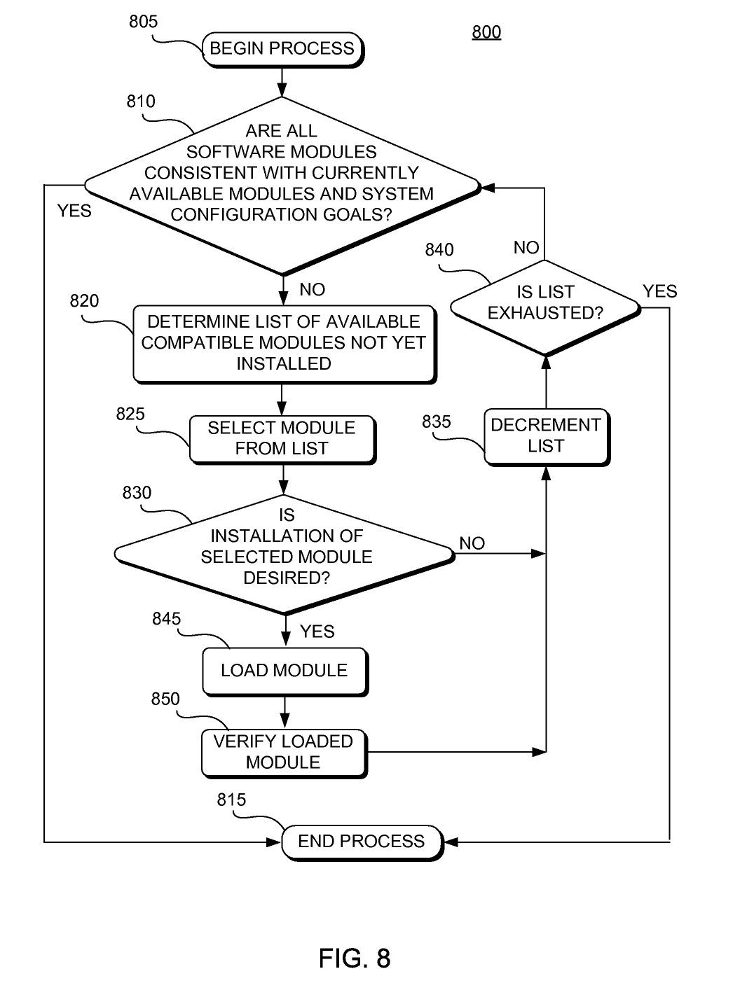

The system 100 of FIG. 1, and the user interfaces 200 through 700 of FIGS. 2 through 7, respectively, facilitate a variety of non-invasive characterizations of hard and soft tissues, and aid in automated rapid, intuitive analysis of such, to derive information useful in determining numerous factors applicable to a broad variety of circumstances generally relevant to intervention with respect to a panoply of presenting medical issues. A process useful in modifying capabilities for displaying such data is described below in Section III, with reference to FIG. 8.

### III. Process

In the previous section, interfacing tools developed in furtherance of functionality with respect to interfacing were disclosed and described. In this section, a process for modification of capabilities of the imaging system is described by reference to a flowchart. Describing the process by reference to one or more flowcharts enables one skilled in the art to develop programs, firmware, or hardware, including such instructions configured to effectuate the process, as well as subsequent revisions, through one or more processors responsive to computer-readable instructions embodied on computer-readable media.

These capacities are often accomplished using suitable computers, including one or more processors, by executing instructions embodied in articles of manufacture such as computer-readable media, or as modulated signals embodied in a carrier wave. As a result, the computer-readable instructions may include capacity for accepting revised computer-readable information descriptive of revised capabilities, which may relate to revisions of aspects of the system 100 via substitution of components, revisions of data-processing structures and the like. Similarly, processes performed by server computer programs, firmware, or hardware also are represented by computer-executable instructions. The process 800 of the present disclosure is implemented by one or more program modules executing on, or performed by, firmware or hardware that is a part of a computer (e.g., computer 130, FIG. 1).

In some embodiments, processes consistent with the subject matter disclosed herein are implementable as a computer data signal embodied in a carrier wave that represents a sequence of instructions which, when executed by one or more processors, such as a processor contained in or associated with the computer 130 in FIG. 1, causes the respective process to occur. As a result, protocols such as those exemplary anatomically-specific characterization procedures described above with reference to Section III may be augmented or revised, for example by downloading suitable software modifications via a network such as a LAN, a WAN, a storage area network, or the Internet, and thus capable of affecting the functionality provided via the image processing module 135 of FIG. 1. Revisions, modifications and the like also may be effectuated via other media suitable for storage, exchange, restoration or augmentation of computer-readable and computer-executable program elements.

In some embodiments, the process 800 disclosed in Section III is implementable via computer-accessible media storing executable instructions capable of directing processor units, such as one or more processors contained in or associated with the computer 130 in FIG. 1, to perform the respective process. In varying embodiments, the medium is a magnetic medium, an electronic medium, or an electromagnetic/optical medium.

More specifically, in a computer-readable program embodiment, programs can be structured in an object-orientation using an object-oriented language such as Java, Smalltalk or C++, and the programs can be structured in a procedural-orientation using a procedural language such as COBOL or C. Software components may communicate in any of a number of ways that are well-known to those skilled in the art, such as application program interfaces (API) or interprocess communication techniques such as remote procedure call (RPC), common object request broker architecture (CORBA), Component Object Model (COM), Distributed Component Object Model (DCOM), Distributed System Object Model (DSOM) and Remote Method Invocation (RMI). The components execute on as few as one computer (e.g., computer 130, FIG. 1), or on multiple computers.

FIG. 8 is a flowchart describing a process 800 capable of utility in the system 100 of FIG. 1. The process 800 of FIG. 8 illustrates one mode for updating of the software for the overall system 100. The process 800 begins in a block 805.

In a query task 810, the process 800 determines when all of the software modules contained in the system 100 are consistent with the collection of presently-available software modules and with the current-applicable configuration goals for the system. A variety of factors may result in a change in either the range of software modules available or in the configuration goals presently desired. For example, addition of new hardware may result in desire to expand the library of protocols in order to realize benefits provided through the revised hardware configuration or upgrade. New surgical procedures and diagnostic tools may give rise to new assessment protocols, or need to coordinate and process an increased range of data types.

Software modification capability allows expansion or modification of a number of predetermined loci representing regions corresponding to specific anatomical features, such as the loci 250, 255 and 260 of FIG. 2. Desire for such may result from modification or retrofitting of the system 100, for example. Revised or new types of image data filter functions may be required or beneficial.

When the query task 810 determines that the software modules presently actualized through the system 100 are consistent with configuration goals and include all relevant software modules and updates, control passes to a block 815, and the process 800 ends. When the query task 810 determines that the software modules presently actualized through the system 100 are not necessarily consistent with configuration goals and or do not necessarily include all relevant software modules and updates, control passes to a block 820.

In the block 820, a list of available software modules that are capable of compatibility in the context of the system 100 and the system configuration goals is prepared. Control then passes to a block 825.

In the block 825, one or more software modules are selected from the list compiled in the block 820. In one embodiment, the software module or modules are selected from a display of a list extracted from the list compiled in the block 820. In one embodiment, a next available example of a software module taken from the list assembled in the block 820 is automatically selected and the selection is displayed to a system maintenance person. Control then passes to a query task 830.

The query task 830 determines when installation of the selected module or modules is desirable. When the query task 830 determines that installation of the selected module or modules is desirable, control passes to a block 845. When the query task 830 determines that installation of the selected module or modules is not desirable, control passes to a block 835.

In the block 835, the list is decremented. In other words, the selected module or modules are removed from the list assembled in the block 820. Control then passes to a query task 840.

The query task 840 determines when the list initially assembled in the block 820 has been exhausted. When the query task 840 determines that the list has been exhausted, control passes to the block 815, and the process 800 ends. When the query task 840 determines that the list has not been exhausted, control passes back to the query task 810 (or to the block 820). One reason for contemplating passing control back to the query task 810 is that as the complement of software modules and capabilities changes with changing software population of the system 100, the implications of compatibilities and needs may change.

For example, a module for comparison of results for two types of analysis would be irrelevant until such point as software modules supporting both of the two types of analysis are present, and that, in turn, may be a function of selections made earlier, in the block 825. This could occur when a hardware modification is capable of supporting more than one mode of operation is being addressed, but only modules corresponding to a portion of those modes are selected for actualization—in that hypothetical situation, it would not be apparent initially that the comparison module might be desired.

When the query task 830 determines that installation of the selected module or modules is desirable, control passes to the block 845. In the block 845, the selected module or modules are loaded or installed. Control then passes to a block 850.

In the block 850, the module or modules that had been loaded in the block 845 are verified. For example, a first check is to ensure that loading was complete and accurate. Also, compatibility of the loaded module or modules, as implemented, with other system elements may need to be verified. Control them passes to the block 835 and the process 800 iterates as described above.

It will be appreciated that the process 800 may be implemented in a number of different ways. For example, a qualified party may supervise downloading of appropriate modules via a modulated carrier wave, such as a signal transmitted via a network such as the Internet. Alternatively, a memory module may be added to the memory 150 of FIG. 1, such as a CD, DVD, or solid state ROM, or a removable data storage device 158 may be coupled to the removable storage device port 156 to download selected data groups as desired.

Accordingly, the process 800 may be updated via addition or substitution of machine-readable and executable instructions in computer-based controllers, as is described above.

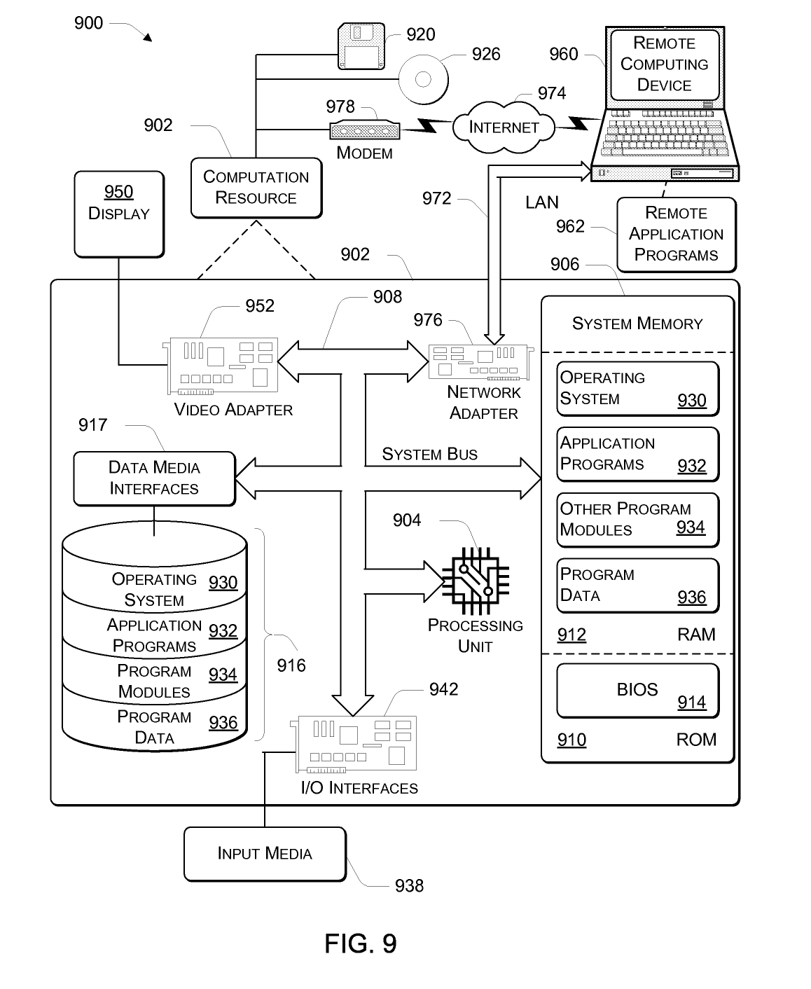

As a result, the system 100 is provided with revised data and instructions. Capabilities of the system 100 are augmented. As an example, a technical effect promoted by such can include capability of transmission, via digital technologies, of radiographic images having improved diagnostic value for immediate contemplation and evaluation by experts during triage, or even during transportation of a victim of an accident from the situs of the disaster to suitable medical facilities—such as during the “golden moments” immediately following determination of injury that are extremely vital to increasing patient survival, as well as recovery trajectory. These features and advantages can represent significant improvements in system performance, from a capabilities perspective as well as reliability considerations. Such enhancements, in terms of machine-controlled performance coordinated in tandem with operator review and approval, may be achieved via the elements described above, as well as in conjunction and cooperation with an operating environment such as that which is described below in Section IV with reference to FIG. 9.

### IV. Hardware and Operating Environment

FIG. 9 illustrates an example of a general computer environment 900 that includes a computation resource 902 capable of implementing the processes described herein. It will be appreciated that other devices may alternatively used that include more components, or fewer components, than those illustrated in FIG. 9.

The illustrated operating environment 900 is only one example of a suitable operating environment, and the example described with reference to FIG. 9 is not intended to suggest any limitation as to the scope of use or functionality of the embodiments of this disclosure. Other well-known computing systems, environments, and/or configurations may be suitable for implementation and/or application of the subject matter disclosed herein.

The computation resource 902 includes one or more processors or processing units 904, a system memory 906, and a bus 908 that couples various system components including the system memory 906 to processor(s) 904 and other elements in the environment 900. The bus 908 represents one or more of any of several types of bus structures, including a memory bus or memory controller, a peripheral bus, an accelerated graphics port and a processor or local bus using any of a variety of bus architectures, and may be compatible with SCSI (small computer system interconnect), or other conventional bus architectures and protocols.

The system memory 906 includes nonvolatile read-only memory (ROM) 910 and random access memory (RAM) 912, which may or may not include volatile memory elements. A basic input/output system (BIOS) 914, containing the elementary routines that help to transfer information between elements within computation resource 902 and with external items, typically invoked into operating memory during start-up, is stored in ROM 910.

The computation resource 902 further may include a non-volatile read/write memory 916, represented in FIG. 9 as a hard disk drive, coupled to bus 908 via a data media interface 917 (e.g., a SCSI, ATA, or other type of interface); a magnetic disk drive (not shown) for reading from, and/or writing to, a removable magnetic disk 920 and an optical disk drive (not shown) for reading from, and/or writing to, a removable optical disk 926 such as a CD, DVD, or other optical media.

The non-volatile read/write memory 916 and associated computer-readable media provide nonvolatile storage of computer-readable instructions, data structures, program modules and other data for the computation resource 902. Although the exemplary environment 900 is described herein as employing a non-volatile read/write memory 916, a removable magnetic disk 920 and a removable optical disk 926, it will be appreciated by those skilled in the art that other types of computer-readable media which can store data that is accessible by a computer, such as magnetic cassettes, FLASH memory cards, random access memories (RAMs), read only memories (ROM), and the like, may also be used in the exemplary operating environment.

A number of program modules may be stored via the non-volatile read/write memory 916, magnetic disk 920, optical disk 926, ROM 910, or RAM 912, including an operating system 930, one or more application programs 932, other program modules 934 and program data 936. A user may enter commands and information into computation resource 902 through input devices such as input media 938 (e.g., keyboard/keypad, tactile input or pointing device, mouse, foot-operated switching apparatus, joystick, touchscreen or touchpad, microphone, antenna etc.). Such input devices 938 are coupled to the processing unit 904 through an input/output interface 942 that is coupled to the system bus (e.g., a serial port interface, a parallel port interface, a universal serial bus (USB) interface, an IEEE 1354 (Firewire) interface, etc.). A monitor 950 or other type of display device is also coupled to the system bus 908 via an interface, such as a video adapter 952.

The computation resource 902 may include capability for operating in a networked environment (as illustrated in FIG. 1, for example) using logical connections to one or more remote computers, such as a remote computer 960. The remote computer 960 may be a personal computer, a server, a router, a network PC, a peer device or other common network node, and typically includes many or all of the elements described above relative to the computation resource 902. In a networked environment, program modules depicted relative to the computation resource 902, or portions thereof, may be stored in a remote memory storage device such as may be associated with the remote computer 960. By way of example, remote application programs 962 reside on a memory device of the remote computer 960. The logical connections represented in FIG. 9 may include a storage area network (SAN, not illustrated in FIG. 9), local area network (LAN) 972 and/or a wide area network (WAN) 974, but may also include other networks.

Such networking environments are commonplace in modern computer systems, and in association with intranets and the Internet. In certain embodiments, the computation resource 902 executes an Internet Web browser program (which may optionally be integrated into the operating system 930), such as the “Internet Explorer” Web browser manufactured and distributed by the Microsoft Corporation of Redmond, Wash.

When used in a LAN-coupled environment, the computation resource 902 communicates with or through the local area network 972 via a network interface or adapter 976. When used in a WAN-coupled environment, the computation resource 902 typically includes interfaces, such as a modem 978, or other apparatus, for establishing communications with or through the WAN 974, such as the Internet. The modem 978, which may be internal or external, is coupled to the system bus 908 via a serial port interface.

In a networked environment, program modules depicted relative to the computation resource 902, or portions thereof, may be stored in remote memory apparatus. It will be appreciated that the network connections shown are exemplary, and other means of establishing a communications link between various computer systems and elements may be used.

A user of a computer may operate in a networked environment 100 using logical connections to one or more remote computers, such as a remote computer 960, which may be a personal computer, a server, a router, a network PC, a peer device or other common network node. Typically, a remote computer 960 includes many or all of the elements described above relative to the computer 900 of FIG. 9.

The computation resource 902 typically includes at least some form of computer-readable media. Computer-readable media may be any available media that can be accessed by the computation resource 902. By way of example, and not limitation, computer-readable media may comprise computer storage media and communication media.

Computer storage media includes volatile and nonvolatile, removable and non-removable media, implemented in any method or technology for storage of information, such as computer-readable instructions, data structures, program modules or other data. The term “computer storage media” includes, but is not limited to, RAM, ROM, EEPROM, FLASH memory or other memory technology, CD, DVD, or other optical storage, magnetic cassettes, magnetic tape, magnetic disk storage or other magnetic storage devices, or any other media which can be used to store computer-intelligible information and which can be accessed by the computation resource 902.

Communication media typically embodies computer-readable instructions, data structures, program modules or other data, represented via, and determinable from, a modulated data signal, such as a carrier wave or other transport mechanism, and includes any information delivery media. The term “modulated data signal” means a signal that has one or more of its characteristics set or changed in such a manner as to encode information in the signal in a fashion amenable to computer interpretation.

By way of example, and not limitation, communication media includes wired media, such as wired network or direct-wired connections, and wireless media, such as acoustic, RF, infrared and other wireless media. The scope of the term computer-readable media includes combinations of any of the above.

The computer 902 may function as one or more of the control segments of module 120 (FIG. 1), the computer 130, the operator console 140 and/or the data acquisition and conditioning module 160, for example, via implementation of the process and 800 of FIG. 8 as one or more computer program modules.

### V. Conclusion

A medical imaging system is described which achieves unified window/level control and that results in user interface complexity reduction. Although specific embodiments have been illustrated and described herein, it will be appreciated by those of ordinary skill in the art that any arrangement which is calculated to achieve the same purpose may be substituted for the specific embodiments shown. This disclosure is intended to cover any adaptations or variations. For example, although described in procedural terms, one of ordinary skill in the art will appreciate that implementations can be made in a procedural design environment or any other design environment that provides the required relationships.

The exemplary user interfaces 200 through 700 allow the clinician to select a modality fitting the clinician's preferences or needs, and which is suitable for the procedure being performed. These user interfaces 200 through 700 each provide process implementations facilitating displaying and evaluating data stored in memory from an examination. The flexibility provided via user selection among multiple viewing modalities for analysis of data that are stored in memory, and the coordination between the plurality of views and formats provided by the image processing module 135 of FIG. 1, streamline the review and characterization of the data.

In particular, one of skill in the art will readily appreciate that the names or labels of the processes and apparatus are not intended to limit embodiments. Furthermore, additional processes and apparatus can be added to the components, functions can be rearranged among the components, and new components to correspond to future enhancements and physical devices used in embodiments can be introduced without departing from the scope of embodiments. One of skill in the art will readily recognize that embodiments are applicable to future communication devices, different file systems, and new data types. The terminology used in this disclosure is meant to include all object-oriented, database and communication environments and alternate technologies which provide the same functionality as described herein.

What is claimed is:

1. A system having a user interface for manipulation of image data in an imaging system, comprising a display region spanning only a locus of interdependent variable values that is exclusive of invalid pairs of parameter values.

2. The system of claim 1, wherein the display region is triangular.

3. The system of claim 1, wherein the imaging system comprises an X-ray imaging system, and the user interface is configured to provide a region of increased resolution that dynamically adjusts responsive to a tactile input device.

4. The system of claim 1, wherein the imaging system comprises an X-ray imaging system, and the display region corresponds to variable values for fitting window/level filtering function parameter values selectable via a touchscreen.

5. The system of claim 1, wherein the display region is bounded by a shape having at least two sides of equal dimension.

6. The system of claim 1, wherein a clinician may interact with the system via a touchscreen for displaying an image within the display region, the image including a plurality of predetermined loci each corresponding to a specific anatomical region and including nonlinear display capability such that a localized portion is displayed at higher resolution than a remainder of the image.

7. The system of claim 1, wherein a first parameter value of a valid parameter pair is a linear function of a second parameter value of the pair.

8. The system of claim 1, wherein the imaging system comprises a fluoroscopic imaging system, and the user interface is configured to provide a region of increased resolution that dynamically adjusts responsive to a tactile input device.

9. The system of claim 1, wherein the imaging system comprises a fluoroscopic imaging system, and the display region corresponds to variable values for fitting window/level filtering function parameter values selectable via a touchscreen.

10. A method for adjusting parameter values of a filtering function employable in forming an image in an imaging system, including providing an image of a user interface for manipulation of image data and providing a display region spanning only a locus of interdependent variable values that is exclusive of invalid groups of parameter values.

11. The method of claim 10, wherein the user interface includes an active region comprising one of a frustum and a triangle.

12. The method of claim 10, wherein the filtering function is a window and level filtering function.

13. The method of claim 10, further including displaying a dynamically-adjustable region within the display region corresponding to an expanded view of a selected portion of the image.

14. The method of claim 10, further including associating an image of a graph with the user interface.

15. The method of claim 10, further including blocking portions of image data corresponding to a user-selectable range of parameter values and forming an image of a region of anatomical interest using image data not including the blocked portions.

16. The method of claim 10, wherein the display region is triangular.

17. An article of manufacture comprising a computer-readable medium having computer-readable instructions embodied thereon, which, when executed by one or more processors, cause the one or more processors to perform acts of:
generating a user interface for manipulation of image data; and
providing a display region within the user interface, spanning only a locus of interdependent variable values that is exclusive of invalid groups of parameter values.

18. The article of manufacture of claim 17, wherein the computer-readable medium comprises a detachable computer-readable medium.

19. The article of manufacture of claim 17, wherein the computer-readable medium comprises a non-volatile memory.

20. The article of manufacture of claim 17, wherein the computer-readable instructions, when executed, cause the one or more processors to perform further acts including:
blocking portions of image data corresponding to a user-selectable range of parameter values; and
forming an image of a region of anatomical interest using image data not including the portions excluded by blocking.

21. The article of manufacture of claim 17, wherein the computer-readable instructions, when executed, cause the one or more processors to perform acts including:
providing one or more image portions together with the image portion representing at least first and second image resolution scaling aspects; and
dynamically adjusting a ratio of scales of the scaling aspects, responsive to user manipulation of a tactile input device.

22. The article of manufacture of claim 17, wherein the article of manufacture is configured to store revised computer-readable instructions supplied from a remote data source.
